(19) United States
(12) Patent Application Publication
(10) Pub. No.: US 2005/0240092 A1
(43) Pub. Date: Oct. 27, 2005

(54) Infusion devices, glucose meters and/or monitors with smell sniffing technology

(75) Inventors: Rajiv Shah, Rancho Palos Verdes, CA (US); Paul H. Kovelman, Simi Valley, CA (US)

Correspondence Address: MEDTRONIC MINIMED, INC., 18000 Devonshire Street, Northridge, CA, 91325-1219, US

(73) Assignee: MEDTRONIC MINIMED, INC.

(21) Appl. No.: 11/111,007

(22) Filed: Apr. 21, 2005

Related U.S. Application Data
(60) Provisional application No. 60/564,771, filed on Apr. 22, 2004.

(51) Int. Cl. G01N 7/00; G01N 33/497; A61B 5/00; A61B 19/00

(52) U.S. Cl. 600/365; 128/897; 73/23.34

(57) ABSTRACT

A medical device system including a medical device, and a smell sniffing technology module. The smell sniffing module is for sniffing parameters associated with a patient and to use information obtained from the sniffing parameters to determine when testing should occur. The medical device is an infusion device, a meter, a blood glucose meter, a monitor, or a glucose monitor. The smell sniffing technology module is incorporated in the medical device, or the smell sniffing technology module is incorporated a separate device from the medical device.

### FIELD OF THE INVENTION

This invention relates to the medical arts and, in particular embodiments, to infusion devices, glucose meters and/or glucose monitors that include smell sniffing technology that permit them to determine when additional testing should be conducted.

### BACKGROUND OF THE INVENTION

Traditionally, diabetic individuals have had to test their blood glucose levels on a regular basis. However, the user is not always aware of the best time to take a blood glucose test. Thus, the user may test a specific times of the day. This is done in the hope of avoiding unnecessary pain associated with drawing blood for the test. An individual could test more often, but this would increase expense (due to extra test strips) and is potentially painful.

Continuous glucose monitoring is a viable option, since it test blood glucose levels on a continuous or near continuous basis. However, not all individuals require such continual monitoring. Also, individuals will not always now the best times to calibrate the blood glucose monitor.

### SUMMARY OF THE DISCLOSURE

According to an embodiment of the invention, a medical device system including a medical device, and a smell sniffing technology module. The smell sniffing module is for sniffing parameters associated with a patient and to use information obtained from the sniffing parameters to determine when testing should occur. In particular embodiments, the medical device is an infusion device. In other embodiments, the medical device is a meter, and may be a blood glucose meter. In still other embodiments, the medical device is a monitor, and may be a glucose monitor. In further embodiments, the smell sniffing technology module is incorporated in the medical device, or the smell sniffing technology module is incorporated a separate device from the medical device.

Other features and advantages of the invention will become apparent from the following detailed description, taken in conjunction with the accompanying drawings which illustrate, by way of example, various features of embodiments of the invention.

### DETAILED DESCRIPTION OF THE PREFERRED EMBODIMENTS

As discussed below for purposes of illustration, the invention is embodied in an infusion device, glucose meter or glucose monitor that includes smell sniffing technology. In preferred embodiments of the present invention, the technology is used to determine the best time to take blood glucose levels in diabetic patients. However, it will be recognized that further embodiments of the invention may be used for other disease states, such as cardiac performance, septus, bacterial or viral infections, or the like, and may be used to determine the best time to test other parameters, such as lactate, pH, oxygen, bacterial or viral load.

The smell sniffing technology can be attached to a medical device, such as an infusion device, a meter (discrete tests), or a monitor (continuous, near continuous or intermittent tests) to augment and improve performance, with the device being selected based on disease type and/or parameter to be measured. In particular embodiments, the infusion device is an external infusion pump that can deliver insulin, such as those manufactured by Medtronic MiniMed, Inc., Deltec Inc., Animas Corporation, Disetronic, or the like. In other embodiments, the meter is a blood glucose meter, such as those manufactured Roche Diagnostics, Johnson & Johnson, Becton Dickinson & Company, Bayer, Abbott Laboratories, TheraSense, or the like. The monitor may be a blood glucose meter, such as the CGMS manufactured by Medtronic MiniMed, Inc., the Biographer by Cygnus, the Navigator by TheraSense, surface mounted sensors proposed by SpectRx, or the like.

The smell sniffing technology is designed to sniff the air or tissue near a patient to determine the presence or absence of particular components or indicators. The technology would determine based on what is sniffed the current health of the patient. For instance, the presence of ketones could indicate DKA in a diabetic patient. The presence of other components could indicate bacterial or viral actions, lack of oxygen in the tissue or changes in pH. Technology of this type is available from Cyrano, Sensobi (Pemstar), or the like.

In particular embodiments, the smell sniffing technology is incorporated into the actual medical device. In other embodiments, the smell sniffing technology is a separate device and may be used separately from the medical device. It may be attachable to the medical device, and it may communicate with the medical device either wirelessly (e.g., RF, IR, Optical, ultrasonic, conduction, etc.) or by wires.

When the presence of a condition that requires testing of a parameter by a meter or monitor is determined. The smell sniffing technology will generate a signal to provide the user with an alarm or notification to perform a test. The alarm or indication can be generated in the smell sniffing device, in the medical device (if the smell sniffing technology is incorporated) or transmitted (by wire or wirelessly) to either a medical device, relay device or remote monitoring station. Data from the smell sniffing technology may be recorded and stored for later use an analysis by a patient and/or medical personnel. Data can be downloaded to a PC or through to a central server by modem, internet, or the like. Reports and analysis can then be performed to determine accuracy and any need for changes for a particular patient or disease state.

The interests with the smell sniffing technology lie in its ability to make standard blood glucose meters more intelligent. Based on analysis of continuous sensor data, 60-75% of finger stick blood glucose measurements from standard blood glucose meters result in no therapeutic response. Individuals tend to sample at the “wrong” time which in turn results in undetected hyperglycemia or hypoglycemia. The standard response to missing hyperglycemia and hypoglycemia is to sample more frequently. In reality most Type 1 diabetics have 4-6 hyperglycemic or hypoglycemic events that require treatment. Sampling a minimum of 12 times per day tends to catch all of the highs and lows and allows therapy in the form of insulin or carbohydrate intake to be applied in a timely manner. However, only the most compliant individual can adhere to such a rigorous and often painful sampling regimen. In an embodiment, the smell sniffing technology is used as a supplement to standard meter technology as opposed to a meter replacement. Surrogate breath chemistries that are correlated to hypoglycemia and hyperglycemia can be assayed hourly as a means of assessing glycemic state. A breathing apparatus plumbed to the Sensobi device could be used, if desired, to deal with periods of sleep. Detected hypoglycemia and hyperglycemia would be translated into an alarm or request to take a finger stick glucose measurement. Treatment is based solely on the blood glucose measurement. Alternatively, some treatment options might be indicated or validated with the smell sniffing technology. Using the smell sniffing technology, the need to take 12 samples per day to achieve optimized blood glucose based on discrete measurements would be reduced to a manageable 4-6 samples per day through the use of a non-invasive device. In other embodiments, the test could be performed more frequently, such as each ½ hour or less frequently, such as every, 2, 4, 6, or 8 hours depending on the needs of the patients. If effectiveness is shown, the need for actual finger sticks could be reduced to 1, 2, 3, 4, 5, or 6 per day. Essentially the smell sniffing technology device becomes the biological clock for blood glucose monitoring, which can provide a significant improvement in glucose control, quality of life, and patient compliance. This also provide the opportunity for improved control, and at the same time a reduction in test strip and supply costs due to savings associated with the efficiency in sampling provided through the smell sniffing technology technology.

Evaluating the smell sniffing technology is straightforward in animals. The protocol would be as follows:

Select several animals with insulin dependent diabetes.

Fast the animals for 12 hours or the overnight period prior to use

Insert a venous access blood sampling catheter in each animal.

Sedate each animal with an appropriate amount of medication.

Place a breathing mask configured with a 3-way valve on the exit tube over the nose and mouth of each animal.

Obtain a baseline signal with a smell sniffing system plumbed to each animal or to a single smell sniffing system that is attached through a manifold to two or more animals. Use of the valve and the tubing length may be optimized to support system purging with nitrogen or something similar.

Obtain venous blood samples every 20 minutes and assay those samples for glucose using calibrated YSI or Chiron blood glucose monitoring instruments. Obtain breath measurements in concert with venous sampling

Glucose can be rapidly manipulated through the distraction of IV glucose or insulin once a stable glucose baseline is obtained. Balancing the insulin and glucose delivered will allow various steady state glucose values to be obtained during the course of the study. We would attempt to manipulate glucose between 50 mg/dl and 350 mg/dl in 100 mg/dl increments. Venous glucose measurements along with corresponding breath measurements will be made during the rapid glucose change. At least 3 blood glucose and breath measurements would be made at each steady state glucose value.

Upon completion of the experiment, smell sniffing technology breath signals would be correlated to venous blood glucose values for each individual animal and for the pooled population. Temporal graphs showing blood glucose and calibrated breath glucose would also be generated. In addition, a continuous blood glucose monitor and sensor could be used as part of the study to see if there are any timing or lag issues associated with the smell sniffing technology data and the current blood glucose levels.

The study would be a success if the following criteria are met:
r=0.8

Clarke Error Grid A&B values>90% for a retrospectively calibrated breath device

Hysterisas or lag of less than 15 minutes when transitioning from high glucose to low glucose values.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

1. A medical device system, comprising:
a medical device; and
a smell sniffing technology module for sniffing parameters associated with a patient and to use information obtained from the sniffing parameters to determine when testing should occur.

2. The system according to claim 1, wherein the medical device is an infusion device.

3. The system according to claim 1, wherein the medical device is a meter.

4. The system according to claim 2, wherein the medical device is a blood glucose meter.

5. The system according to claim 1, wherein the medical device is a monitor.

6. The system according to claim 5, wherein the medical device is a glucose monitor.

7. The system according to claim 1, wherein the smell sniffing technology module is incorporated in the medical device.

8. The system according to claim 1, wherein the smell sniffing technology module is incorporated a separate device from the medical device.
